(19) United States
(12) Patent Application Publication
(10) Pub. No.: US 2006/0094780 A1
(43) Pub. Date: May 4, 2006

(54) Novel antibiotic compounds

(76) Inventors: Rodney Warren Rickards, Weetangera (AU); Chunjiu Zhao, Changchun (CN); Stephen Charles Trowell, Oxley (AU)

Correspondence Address: GREENLEE WINNER AND SULLIVAN P C, 4875 PEARL EAST CIRCLE, SUITE 200, BOULDER, CO, 80301, US

(21) Appl. No.: 11/256,494

(22) Filed: Oct. 21, 2005

Related U.S. Application Data
(63) Continuation-in-part of application No. 10/296,375, filed on May 22, 2003.

(30) Foreign Application Priority Data
May 22, 2000 (AU) PQ7653
Feb. 4, 2003 (AU) 2003900555

(51) Int. Cl. A61K 31/22; A61K 31/225; A61K 31/045

(52) U.S. Cl. 514/546; 514/548; 514/729

(57) ABSTRACT

The present invention relates to the determination that a group of trinervitadiene compounds possess antimicobial activity. The invention provides pharmaceutical and/or veterinary formulations comprising these compounds, as well as methods for treating a microbial infection or disease, and methods for disinfecting a surface.

### CROSS REFERENCE TO RELATED APPLICATIONS

This application is a continuation-in-part of U.S. patent application Ser. No. 10/296,375, filed May 22, 2003 and claims priority from PCT/AU2004/000123, filed Feb. 3, 2004, which in turn claims priority from Australian Patent Application 2003900555, filed Feb. 4, 2003. U.S. patent application Ser. No. 10/296,375 is the national phase application of Patent Cooperation International Application PCT/AU01/00589, filed May 22, 2001, which claims priority from Australian Patent application PQ7653, filed May 22, 2000. All of the priority applications are incorporated herein by reference in their entirety to the extent not inconsistent herewith.

### BACKGROUND OF THE INVENTION

This invention relates to a novel class of antibiotic compounds and their use for treatment of various microbial infections and diseases in humans and other animals.

Antibiotics, compounds with selective toxicity against infectious microorganisms, present humanity with enormous benefits and are credited with saving many millions of lives since their introduction in the 20th century. Today there is a continuing need for new antibiotics to assist in the management of multiply resistant pathogens (e.g. multiply resistant Staphyloccus aureus or vancomycin-resistant enterococcus) or to provide improved therapies for difficult-to-treat pathogens such as Mycobacterium tuberculosis, the causative agent of tuberculosis). Selectively toxic compounds also have utility as veterinary antibiotics and growth enhancers, where there is a need to develop agents with different modes of action from those used in humans, and also as preservatives and antisepsis agents in a wide range of medical and industrial processes and products.

Insects and terrestrial invertebrates face infection by many opportunistic microbial pathogens, yet they are a successful group of organisms which have been present on earth for hundreds of millions of years and are today represented by many millions of species, far more than any other group of macroorganisms. Insects and other terrestrial invertebrates must therefore have efficient methods for avoiding or overcoming potential infections.

Insects share with mammals and other organisms an “innate” immune system based on non-specific phagocytosis of foreign material by haemocytes, and production of a range of antimicrobial peptides such as defensins, cecropins and attacins in response to general microbial inducers such as lipopolysaccharide and (1,3)-beta-D-glucans. However, there has been no evidence from insects, or any other invertebrate, for the presence of a clonal, inducible-immune system of the B-lymphocyte/T-lymphocyte type that typifies mammalian responses to infection. Insects may therefore have other, undiscovered, defensive systems to protect themselves against microbial invasion.

There has been little previous evidence for the synthesis of non-peptide antibiotics by insects. A survey of 102 species of North American arthropods in the 1950's (DeCoursey, Webster et al. 1953) revealed only two active extracts, and these were presumed to be active due to the presence of quinones, reactive compounds of no value as antibiotics. An antibacterial compound, para-hydroxycinnamaldehyde has recently been isolated from a Korean sawfly (Leem, Jeong et al. 1999), however no data on the mammalian toxicity of this compound was presented.

Between 1997 and 1999, the present applicants assembled a large collection of terrestrial invertebrates from the east coast of Australia and extracted a number of them and screened the extracts for biological activity. One particular extract from an Australian species of termite, Nasutitermes triodiae (Isoptera:Termitidae) (Froggatt), was shown to have antimicrobial activity against the Gram positive organism Bacillus subtilis. The extract was also shown to have only intermediate levels of growth-inhibitory activity against two transformed mammalian cells, namely SP₂/O—Ag8 a non-secreting mouse myeloma cell line derived from Balb/C mice, and NCl-H460 a human-derived small cell lung carcinoma cell line.

Four compounds have been purified to homogeneity from the extract of N. triodiae. These were a triol of a trinervitadiene (Formula (6)); a monoacetate of the same triol (Formula (8)); and two diols with the same trinervitadiene carbon skeleton (Formulae (7) and (9)). In addition, the triacetate (Formula (10)) of the aforementioned triol was synthesised by esterification with acetic anhydride. Of these compounds, all but one of the diols (9) represents a previously unreported structure. Furthermore, all of the compounds had measurable antimicrobial activity, a property not previously reported for any trinervitadiene. The triol (6) was shown to have moderately good antimicrobial potency against the target organism. The novel diol (7) had similar antimicrobial potency to the triol, while the known diol (9) was 2-4 times more potent. The monoacetate and triacetate were also both active, albeit less potent than the diols or triol. The triol (6) was also tested in mammalian cell culture and shown to have selective toxicity for the test microorganism over mammalian cells.

These trinervitadiene compounds therefore have potential utility as human or veterinary antibiotics or as antiseptic agents in industrial or other processes. Furthermore, because the results provided herein demonstrate for the first time that derivatives of the trinervitadiene carbon skeleton have antimicrobial properties, it may reasonably be concluded that other derivatives of this carbon skeleton will also have similar selective antimicrobial properties.

### SUMMARY OF THE INVENTION

Thus, in a first aspect, the present invention provides a method for treating a microbial infection or disease in a subject, said method comprising administering to said subject an effective amount of a compound according to the formula: 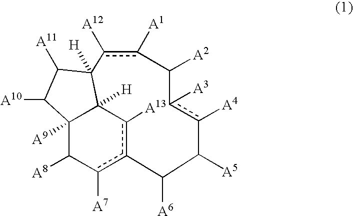
wherein;

Z,1 denotes a single or double bond or an epoxidised bond, and

(i) substituents A¹ to A¹³ are selected, independently, from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy, or

(ii) any one or more of substituent pairs A¹ and A², A¹ and A³, A² and A³, A² and A⁴, A³ and A⁴, A³ and A⁵, A⁴ and A⁵, A⁴ and A⁶, A⁵ and A⁶, A⁶ and A⁷, A⁷ and A⁸, A⁷ and A⁹, A⁸ and A⁹, A⁸ and A¹⁰, A⁹ and A¹⁰, A⁹ and A¹¹, A¹⁰ and A¹¹, A¹¹ and A¹², A¹ and A¹², and A² and A¹² form a substituted or unsubstituted heterocyclic group, wherein any substituents, including A¹³, not forming a substituted or unsubstituted heterocyclic ring, are selected independently from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy;

with the provisos that,

only one of the bonds between C1 and C2, and C1 and C15, may be a double bond or epoxide,

when the bond between C1 and C2 is a double bond or epoxide, A⁷is bound to C2 by a single bond,

when the bond between C1 and C15 is a double bond or epoxide, A¹³ is bound to C15 by a single bond,

when the bond between C8 and C9 is a double bond or epoxide, A¹ and A¹² are bound to C9 and C8 respectively by a single bond, and

when the bond between C11 and C12 is a double bond or epoxide, A³ and A⁴ are bound to C11 and C12 respectively by a single bond;

and pharmaceutically/veterinary-acceptable salts thereof.

The term “lower” is intended to mean a group having 1 to 6 carbon atom(s), unless otherwise provided.

Suitable “lower alkyl” and lower alkyl moieties in the terms “lower alkoxy”, “lower alkythio”, “lower alkylamino”, “lower alkylsulfonyl”, “lower alkylsulfinyl” and “lower alkylsulfonyloxy” may be straight or branched such as methyl, ethyl, propyl, isopropyl, butyl, isobutyl, tert-butyl, pentyl, hexyl or the like.

Suitable “lower alkene” groups may be CH₂, CHCH₃, CHCH₂, CHCHCH₃ and the like. Similarly, suitable “lower alkyne” groups may be CH, CCH₃, CCH, CCCH₃ and the like.

Suitable “lower alkoxy” may be methoxy, ethoxy, propoxy, isopropoxy, butoxy, isobutoxy, tert-butoxy and the like.

Suitable “lower carboxy” may be carboxymethyl, carboxyethyl, carboxypropyl, carboxyisopropyl, carboxybutyl, carboxyisobutyl, carboxy tert-butyl and the like.

A suitable “lower aldehyde group” may be selected from aldehyde groups such as methanal, ethanal, propanal, isopropanal, butanal, isobutanal, tert-butanal and the like.

A suitable “lower ketone group” may be selected from ketone groups such as methanone, ethanone, propanone and the like.

A suitable “lower ester group” may be methanoate, ethanoate, propanoate, isopropanoate, butanoate, isobutanoate, tert-butanoate and the like.

A suitable “lower acyloxy group” may be acetoxy, propionyloxy, butyryloxy and the like.

A suitable “lower alcohol group” may be methanol, ethanol, propanol, isopropanol, butanol, isobutanol, tert-butanol and the like.

Suitable “lower alkylthio” include methylthio, ethylthio, propylthio, butylthio and the like, and lower alkyl thio substituted lower alkyl such as methylthiomethyl, methylthioethyl, methylthiopropyl, methylthiobutyl, ethylthiomethyl, ethylthioethyl, ethylthiopropyl, ethylthiobutyl and the like.

Suitable “lower alkylamino” include methylamino, ethylamino, propylamino, butylamino and the like, and mono or di(lower alkyl) amino substituted lower alkyl such as methylaminomethyl, methylaminoethyl, methylaminopropyl, methylaminobutyl, ethylaminomethyl, ethylaminoethyl, ethylaminopropyl, ethylaminobutyl, dimethylaminomethyl, dimethylaminoethyl, dimethylaminopropyl, dimethylaminobutyl, diethylaminomethyl, diethylaminoethyl, diethylaminopropyl, diethylaminobutyl and the like.

Suitable “lower alkylsulfonyl” may be methylsulfonyl, ethylsulfonyl, propylsulfonyl, butylsulfonyl and the like.

Suitable “lower alkylsulfinyl” include methylsulfinyl, ethylsulfinyl, propylsulfinyl, butylsulfinyl and the like.

Suitable “lower alkylsulfonyloxy” include methylsulfonyloxy ethylsulfonyloxy, propylsulfonyloxy, butylsulfonyloxy and the like.

Suitable substituted or unsubstituted heterocyclic groups may be groups having a carbon and oxygen backbone of 5 to 8 atoms (inclusive of the 2-3 carbon atoms contributed by the Formula (1) structure), including cyclic acetals and cyclic carbonates. Such heterocyclic groups may be substituted by one or more of OH, O, SH, NH₂, lower alkyl, lower alkene, lower alkyne, lower alkoxy, lower carboxy, lower aldehyde groups, lower ketone groups, lower ester groups, lower acyloxy, lower alcohol groups, lower alkylthio, lower alkylamino, lower alkysulfonyl, lower alkysulfinyl and lower alkylsulfonyloxy.

Preferably, A¹, A², A³, A⁵, A⁶, A⁷, A⁸, A¹⁰ and A¹¹ are selected, independently, from H, OH, O, SH, NH₂ and OR. More preferably, A¹, A², A³, A⁵, A⁶, A⁷, A⁸, A¹⁰ and A¹¹ are selected, independently, from H, OH and OR. R in the group OR is a lower alkyl as defined above (preferably, methyl or ethyl) or lower acyl.

Preferably, A⁴, A⁹ and A¹³ are selected, independently, from lower alkyl, lower carboxy, lower aldehyde groups, lower ketone groups, lower ester groups, lower acyloxy and lower alcohol groups. More preferably, A⁴ and A¹³ are selected, independently, from methyl, methanoate and methanol groups, and A⁹ is selected from methanol and CH₂OR groups. Again, R in the group OR is a lower alkyl as defined above (preferably, methyl or ethyl) or lower acyl.

Preferably, A¹² is selected from lower alkyl, lower alkene or lower alkyne. More preferably, A¹² is selected from methyl and CH₂. Most preferably, A¹² is CH₂.

It is also preferred that at least two of said A¹ to A¹³ consist or comprise OH or OR groups, wherein R is as defined above.

Suitable pharmaceutically/veterinary-acceptable salts of the compound of formula (1) include non-toxic salts such as acid addition salts such as aninorganic acid addition salt (e.g. hydrochloride, sulfate, phosphate, etc.), an organic acid addition salt (e.g. formate, acetate, trifluoroacetate, etc.), a salt with an amino acid (e.g. arginine salt, etc.), a metal salt such as an alkali metal salt (e.g. sodium salt, potassium salt, etc.) and an alkaline earth metal salt (e.g. calcium salt, magnesium salt, etc.), an ammonium salt, an organic base addition salt (e.g. trimethylamine salt, triethylamine salt, etc.) and the like.

Preferably, the compound used in the method of the present invention is of the formula: 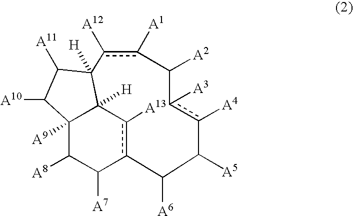
wherein;

[symbol] denotes a single or double bond or an epoxidised bond, and

substituents A¹ to A¹³ are selected, independently, from H, OH, O, lower alkyl, lower alkene, lower alkoxy, lower carboxy, lower aldehyde groups, lower ketone groups, lower ester groups, lower acyloxy and lower alcohol groups;

with the provisos that,

when the bond between C1 and C15 is a double bond or epoxide, A¹³ is bound to C15 by a single bond,

when the bond between C8 and C9 is a double bond or epoxide, A¹ and A¹² are bound to C9 and C8 respectively by a single bond, and

when the bond between C11 and C12 is a double bond or epoxide, A³ and A⁴ are bound to C11 and C12 respectively by a single bond;

and pharmaceutically/veterinary-acceptable salts thereof.

More preferably, the compound used in the method of the present invention is of the formula: 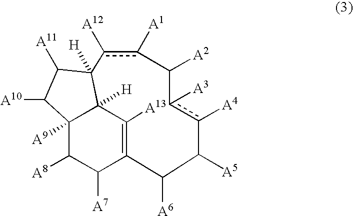
wherein;

[symbol] denotes a single or double bond or an epoxidised bond, and

substituents A¹ to A¹³ are selected, independently, from H, OH, O, methyl, ethyl, propyl, butyl, methene, ethene and propene groups, methanal, ethanal, propanal, butanal, methanone, ethanone and propanone groups, methanoate, ethanoate, propanoate and butanoate groups, acetoxy, propionyloxy and butyryloxy groups, and methanol, ethanol, propanol and butanol groups;

with the provisos that,

when the bond between C8 and C9 is a double bond or epoxide, A¹ and A¹² are bound to C9 and C8 respectively by a single bond, and

when the bond between C11 and C12 is a double bond or epoxide, A³ and A⁴ are bound to C11 and C12 respectively by a single bond;

and pharmaceutically/veterinary-acceptable salts thereof.

Even more preferably, the compound used in the method of the present invention is of the formula: 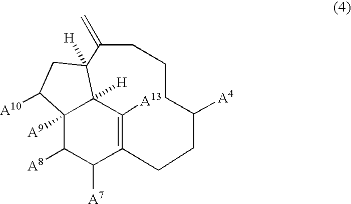
wherein;

substituents A⁴, A⁷, A⁸, A⁹, A¹⁰ and A¹³ are as defined above in relation to formula (1),

and pharmaceutically/veterinary-acceptable salts thereof;

or: 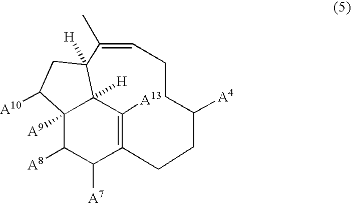
wherein;

substituents A⁴, A⁷, A⁸, A⁹, A¹⁰ and A¹³ are as defined above in relation to formula (1),

and pharmaceutically/veterinary-acceptable salts thereof.

Most preferably, the compound used in the method of the present invention is selected from;

1(15),8(19)-Trinervitadiene-3α,5α, 18-triol,

1(15),8(19)-Trinervitadiene-3α,5α-diol,

1(15),8(19)-Trinervitadiene-3α,5α, 18-triol 5-acetate,

1(15),8(9)-Trinervitadiene-2β,3α-diol, and

1(15),8(19)-Trinervitadiene-3α,5α,18-triol 3,5,18-triacetate.

The compounds defined herein have antimicrobial activity and are therefore useful as, for example, human or veterinary or aquatic antibiotics, as antiseptic/disinfectant agents in industrial or other processes, as agricultural chemicals (including those compositions which can be applied to plants to control microbial infections), or as food preservatives. Further applications include, but are not limited to, inhibition of growth of microbial pathogens in environmental situations, reduction or prevention of microbial colonisation of medical media including washing solutions, ointments and the like.

Also provided is a method of treating/(disinfecting and cleaning) medical indwelling devices comprising administering a composition comprising an effective amount of a compound of the invention. These devices may advantageously include, for example, any indwelling device, for example catheters, orthopedic devices and implants.

The compounds according to the present invention can be used against a broad range of microorganisms causing various infectious diseases and are effective to prevent, alleviate or cure diseases caused by these pathogens.

Examples of bacteria or bacterium-like microorganisms on which the compounds of the invention are effective include, but are not limited to, bacilli such as Bacillus subtilis, Bacillus anthracis, Bacillus cereus; staphylococci such as Streptococcus pyogenes, Streptococcus haemolyticus, Streptococcus faecalis, Streptococcus pneumoniae, Staphylococcus aureus; peptostreptococci such as Neisseria gonorrhoeae, Citrobacter sp., Shigella sp., Klebsiella pneumoniae, Enterobacter sp., Serratia sp., Proteus sp., Pseudomonas aeruginosa, Haemophilus influenzae, Acinetobacter sp., Campylobacter sp., and Chlamydia sp. such as Chlamydia trachomatis and Chlamydia pneumoniae. In a preferred embodiment, the microbe is a Gram-positive bacteria.

In another embodiment, the microbe is a Gram-negative bacteria but not Escherichia sp.

The methods and formulations of the invention may be used for the treatment or prevention of an microbial infection or disease selected from, for example, bacterial infection of wounds including surgical wounds, lung infections (e.g. tuberculosis), skin infections, and systemic bacterial infections. For instance, diseases which can be treated or prevented by the antimicrobial compounds of the present invention include, but are not limited to, anthrax, food poisoning, folliculitis, furuncle, carbuncle, erysipelas, phlegmon, lymphangitis/lymphadenitis, felon, subcutaneous abscess, spiradenitis, acne agminata, infectious atheroma, perianal abscess, masitadenitis, superficial secondary infections after trauma, burn or surgery trauma, pharyngolaryngitis, acute bronchitis, tonsillitis, chronic bronchitis, bronchiectasis, diffuse panbronchiolitis, secondary infections of chronic respiratory diseases, pneumonia, pyelonephritis, cystitis, prostatitis, epididymitis, gonococcal urethritis, non-gonococcal urethritis, cholecystitis, cholangitis, bacillary dysentery, enteritis, adnexitis, intrauterine infections, bartholinitis, blepharitis, hordeolum, dacryocystitis, tarsadenitis, keratohelcosis, otitis media, sinusitis, paradentosis, pericoronitis, gnathitis, peritonitis, endocarditis, septicemia, meningitis, and skin infections.

The compounds of the present invention are also effective on various microorganisms causing veterinary diseases, such as those belonging to the genera Salmonella, Pasteurella, Haemophilus, Bordetella, Staphylococcus, and Mycoplasma. Illustrative examples of the veterinary diseases include those of fowl, such as colibacillosis, pullorum disease, avian paratyphosis, fowl cholera, infectious coryza, staphylomycosis, and mycoplasmosis; those of pigs, such as colibacillosis, salmonellosis, pasteurellosis, hemophilus infections, atrophic rhinitis, exudative epidermitis, and mycoplasmosis; those of cattle, such as colibacilosis, salmonellosis, hemorrhagic septicemia, mycoplasmosis, bovine contagious pleuropneumonia, and bovine mastitis; those of dogs, such as colisepsis, salmonellosis, hemorrhagic septicemia, pyometra, and cystitis; those of cats, such as exudative pleurisy, cystitis, chronic rhinitis, and hemophilus infections; and those of kittens, such as bacterial diarrhea and mycoplasmosis.

The invention also provides a method for treatment and/or prophylaxis of parasitic infections, particularly those caused by protozoan parasites. Included among the protozoan parasites are those of the genera Giardia, Trichomonas, Leishmania, Trypanosoma, Crithidia, Herpetomonas, Leptomonas, Histomonas, Eimeria, Isopora and Plasmodium. An example of a parasitic infection caused by Plasmodium is malaria.

The invention also provides a method for treatment and/or prophylaxis of fungal infections. Such fungal infections (mycoses) may be, for example, cutaneous, subcutaneous or systemic. Superficial mycoses include tinea capitis, tinea corporis, tinea pedis, onychomycosis, perionychomycosis, pityriasis versicolor, oral thrush, and other candidoses such as vaginal, respiratory tract, biliary, eosophageal, and urinary tract candidoses. Systemic mycoses include systemic and mucocutaneous candidosis, cryptococcosis, aspergillosis, mucormycosis (phycomycosis), paracoccid ioidomycosis, North American blastomycosis, histoplasmosis, coccidioidomycosis, and sporotrichosis. Fungal infections include those caused by Cladosporium cucumerinum, Epidermophyton floccosum, Aspergillus fumigatus, and other Aspergillus spp., Rhizopus spp. and Microspermum ypseum.

The method of the invention may be for the treatment of an antimicrobial infection or disease selected from, for example, bacterial infection of wounds including surgical wounds, lung infections (e.g. tuberculosis), skin infections, and systemic bacterial infections.

For pharmaceutical and/or veterinary applications, the compound or pharmaceutically/veterinary-acceptable salt thereof, is formulated for administration by any of the commonly used routes such as oral, nasal, rectal, vaginal, intramuscular, intraveneous administration routes. For convenience, it is preferred that the compound is formulated for oral administration, wherein the compound or pharmaceutically/veterinary-acceptable salt thereof may be in admixture with commonly known binding materials and excipients. Suitable oral formulations may be in the form of capsules, tablets, caplets or syrups.

As will be recognised by those skilled in the art the compounds defined herein can be usefully incorporated in a varied range of formulations/compositions. For example, the compounds can be formulated for administration into an animal, including humans, or incorporated in a range of personal care products including body care and oral care such as deodorants, soaps, shampoos, dentifrices, mouthwashes etc. Suitable formulations for use in the methods of the invention can readily be prepared by the skilled addressee using standard procedures.

The present invention also provides for a cleaning composition for cleaning surfaces, for example hard surfaces, woven or unwoven surfaces. Examples of surfaces which may be cleaned and/or cleaning compositions of the invention include toilet bowls, bath tubs, drains, high chairs, countertops (such as those exposed to meats, vegetables), meat processing rooms, butcher shops, airducts, airconditioners, carpets, paper or woven product treatment, diapers and healthy air machines.

The cleaning product may be in the form of a toilet drop-in for prevention and removal of soil and under rim cleaner for toilets.

In another embodiment the compositions will find application as washing solutions, particularly in contact lens cleaning compositions. Thus contact lenses can be cleaned and disinfected by administering a composition comprising an effective amount a compound defined herein.

The compounds defined herein may be incorporated into personal care products and skin care products. The compounds defined herein may be used in the preparation of epidermal bandages and lotions. In an alternative embodiment, the compounds defined herein may be incorporated into, for example, aftershaves or lotions.

A pharmaceutical and/or veterinary formulation of the invention comprises suitable “excipients”, also referred to as “carreiers”, such that the formulation can be administered to an animal, preferably a human.

Also provided are compositions which can be applied to plants without having a detrimental affect upon the plant.

In another aspect, the composition may comprise a suitable diluent wherein the composition may actually be toxic to, for example, an animal. Such compositions can be useful in industrial settings for disinfecting surfaces etc. Suitable diluents would be well known by those skilled in the art.

In one embodiment, the compounds defined herein are formulated as an emulsion. Emulsions are finely divided or colloidal dispersions comprising two immiscible liquids or “phases”, e.g. oil and water, one of which (the internal or discontinuous phase) is dispersed as droplets within the other (external or continuous phase). Thus, an oil-in-water emulsion consists of oil as the internal phase and water as the external or continuous phase, the water-in-oil emulsion being the opposite.

A wide variety of emulsified systems may be formed comprising a compound defined herein and using microfluidizing technology including standard emulsions and microemulsions.

Generally, emulsions comprise oil and water phases, emulsifiers, emulsion stabilizers, and optionally thickening agents, preservatives, colouring agents, flavouring agents, pH adjusters and buffers, chelating agents, vitamins, anti-foam agents, tonicity adjusters and anti-oxidants. Suitable emulsifiers include (wherein bracketed numerals refer to the preferred hydrophile-lipophile balance (HLB) value): anionic surfactants such as alcohol ether sulfates, alkyl sulfates (30-40), soaps (12-20) and sulfosuccinates; cationic surfactants such as quarternary ammonium compounds; zwitterionic surfactants such as alkyl betaine derivatives; amphoteric surfactants such as fatty amine sulfates, difatty amine sulfates, difatty alkyl triethanolamine derivatives (16-17); and nonionic surfactants such as the polyglycol ether derivatives of aliphatic or cycloaliphatic alcohols, saturated fatty acids and alkylphenols, water-soluble polyethyleneoxy adducts onto polypropylene glycol and alkyl polypropylene glycol, nonylphenol polyoxyethanols, castor oil polyglycol ethers, polypropylene/polyethylene oxide adducts, tributylphenoxy-polyethoxyethoxy- ethanol, lanothin alcohols, polyethylated (POE) alkyl phenols (12-13), POE fatty esters poloxamers (7-19), POE glycol monoethers (13-16), polysorbates (17-19) and sorbitan esters (2-9). This list is not intended to be exhaustive as other emulsifiers are suitable.

In another embodiment, a compound defined herein is formulated in an aqueous composition comprising a water miscible solvent. Examples, of such water miscible solvents include, but are not limited to, ethanol, isopropanol, diethylene glycol monomethyl ether, diethylene glycol butyl ether, diethylene glycol monoethyl ether, diethylene glycol dibutyl ether, polyethylene glycol-300, polyethylene glycol-400, propylene glycol, glycerine, 2-pyrrolidone, N-methyl 2-pyrrolidone, glycerol formal, dimethyl sulfoxide, dibutyl sebecate, polysorbate 80, and mixtures thereof.

Dosage forms of pharmaceutical preparations containing a compound of the present invention are appropriately selected according to the administration route and can be prepared by conventional preparation methods. Typically, the compound is formulated for administration by any of the commonly used routes such as oral, nasal, rectal, vaginal, intramuscular, intraveneous administration routes.

For convenience, it is preferred that for human or veterinary uses the compound is formulated for oral administration, wherein the compound or pharmaceutically/veterinary-acceptable salt thereof may be in admixture with commonly known binding materials and excipients. Examples of dosage forms for oral administration include tablets, powders, granules, capsules, solutions, syrups, elixirs, and oily or aqueous suspensions. The compound can be administered to animals orally either directly or by mixing with feedstuff, or in a dissolved form directly given to animals or by mixing with water or feedstuff.

Injectable preparations may contain adjuvants, such as stabilizers, antiseptics, and solubilizers. The injectable solution which may contain these adjuvants may be put into a container and solidified by, for example, lyophilization to prepare a solid preparation which is dissolved on use. The container may contain either a single dose or multiple doses.

Preparations for external application include solutions, suspensions, emulsions, ointments, gels, creams, lotions, and sprays.

Solid preparations may contain, in addition to the active compound, pharmaceutically acceptable additives. For example, the active compound is mixed with additives selected according to necessity from among fillers, extenders, binders, disintegrators, absorption accelerators, wetting agents, and lubricants and formulated into solid preparations.

Liquid preparations include solutions, suspensions, and emulsions. They may contain adjuvants, such as suspending agents, emulsifiers, and so forth.

For veterinary use, the compound can be formulated into powders, fine granules, soluble powders, syrups, solutions, and injections according to the customary methods in the art.

Typically, the compound or pharmaceutically/veterinary-acceptable salt thereof, will be administered at an effective antimicrobial amount, such as 1 to 100 mg/kg, preferably 5 to 20 mg/kg. For use as drugs for humans, the dose of the compound can be in the range of from 1 mg to 1 g, and preferably from 100 mg to 300 mg, per day for an adult.

For veterinary use, the dose is generally in the range of from 1 to 200 mg, and preferably from 5 to 100 mg, per kg of body weight per day while varying depending on the purpose of administration (for therapy or for prevention), the kind and the size of the animal, the kind of the pathogenic organisms, and severity of symptom.

The above-mentioned daily doses can be given once a day or in 2 to 4 divided doses. If necessary, a daily dose may exceed the above-specified range.

Accordingly, in a second aspect, the present invention provides a pharmaceutical and/or veterinary formulation for treating a microbial infection or disease in a subject, said formulation comprising a compound according to any of the formulae (1) to (5) in admixture with a suitable pharmaceutically/veterinary-acceptable excipient.

The compound of any of the formulae (1) to (5) may also be useful for other non-pharmaceutical/veterinary uses such as in disinfectants and cleaners.

Thus, in a third aspect, the present invention provides a method for disinfecting a surface (e.g. a hard surface such as kitchen bench tops, bathroom tiles and the like), said method comprising applying to said surface an amount of a compound according to the formula: 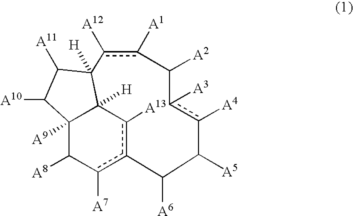
wherein;

[symbol] denotes a single or double bond or an epoxidised bond, and

(i) substituents A¹ to A¹³ are selected, independently, from H, OH, O, SH, NH₂, lower alkyl, lower alkene, lower alkyne, lower alkoxy, lower carboxy, lower aldehyde groups, lower ketone groups, lower ester groups, lower acyloxy, lower alcohol groups, lower alkylthio, lower alkylamino, lower alkysulfonyl, lower alkysulfinyl and lower alkylsulfonyloxy, or

(ii) any one or more of substituent pairs A¹ and A², A¹ and A³, A² and A³, A² and A⁴, A³and A⁴, A³and A⁵, A⁴and A⁵, A⁴and A⁶, A⁵ and A⁶, A⁶ and A⁷, A⁷and A⁸, A⁷ and A⁹ , A⁸ and A⁹, A⁸ and A¹⁰, A⁹ and A¹⁰, A⁹ and A¹¹, A¹⁰ and A¹¹,A¹¹ and A¹², A¹and A¹², and A² and A¹² form a substituted or unsubstituted heterocyclic group, wherein any substituents, including A¹³, not forming a substituted or unsubstituted heterocyclic ring, are selected independently from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy;

with the provisos that,

only one of the bonds between C1 and C2, and C1 and C15, may be a double bond or epoxide,

when the bond between C1 and C2 is a double bond or epoxide, A⁷ is bound to C2 by a single bond,

when the bond between C1 and C15 is a double bond or epoxide, A¹³ is bound to C15 by a single bond,

when the bond between C8 and C9 is a double bond or epoxide, A¹ and A¹² are bound to C9 and C8 respectively by a single bond, and

when the bond between C11 and C12 is a double bond or epoxide, A³ and A⁴ are bound to C11 and C12 respectively by a single bond;

and salts thereof.

In a fourth aspect, the present invention provides an antimicrobial compound of the formula: 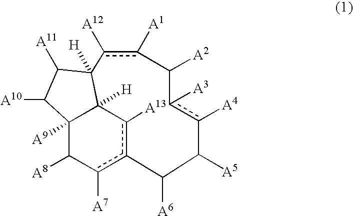
wherein;

[symbol] denotes a single or double bond or an epoxidised bond, and

(i) substituents A¹ to A¹³ are selected, independently, from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy, or

(ii) any one or more of substituent pairs A¹ and A², A¹ and A³, A² and A³, A² and A⁴, A³ and A⁴, A³ and A⁵, A⁴ and A⁵, A⁴ and A⁶, A⁵ and A⁶, A⁶ and A⁷, A⁷ and A⁸, A⁷ and A⁹, A⁸ and A⁹, A⁸ and A¹⁰, A⁹ and A¹⁰, A⁹ and A¹¹, A¹⁰ and A¹¹, A¹¹ and A², A¹ and A¹², and A² and A¹² form a substituted or unsubstituted heterocyclic group, wherein any substituents, including A¹³, not forming a substituted or unsubstituted heterocyclic ring, are selected independently from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy;

with the provisos that,

only one of the bonds between C1 and C2, and C1 and C15, may be a double bond or epoxide,

when the bond between C1 and C2 is a double bond or epoxide, A⁷ is bound to C2 by a single bond,

when the bond between C1 and C15 is a double bond or epoxide, A¹³ is bound to C15 by a single bond,

when the bond between C8 and C9 is a double bond or epoxide, A¹ and A¹² are bound to C9 and C8 respectively by a single bond, and

when the bond between C11 and C12 is a double bond or epoxide, A³ and A⁴ are bound to C11 and C12 respectively by a single bond;

and salts thereof, with the further proviso that said compound is not 1(15),8(9)-Trinervitadiene-2β,3α-diol.

Preferably, the compound of the fourth aspect is in a substantially purified form.

In a fifth aspect, the present invention provides an antimicrobial trinervitadiene compound in a substantially purified form, said compound being obtainable from a termite of the genus Nasutitermes.

### BRIEF DESCRIPTION OF THE DRAWINGS

FIG. 1 shows examples of other trinervitadiene derivatives where the number, position and nature of groups is varied with respect to compounds 6-10. These compounds are expected to have utility as antibiotics. Alternatively, they may be useful lead compounds for the development of derivatives with enhanced antibiotic activities.

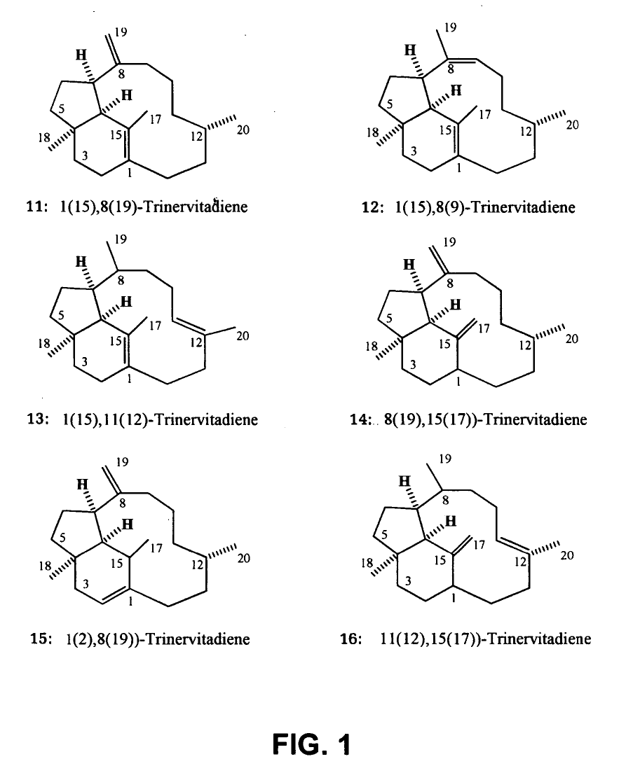

### DETAILED DESCRIPTION OF THE INVENTION

At least some of the compounds of the present invention are naturally occurring trinervitanes which have been obtained from native Australian termites, and are likely to be present in the same and possibly in related species in other countries. Access to larger quantities of these and related natural trinervitanes, which would be needed for therapeutic use, could be obtained from cultured colonies of the appropriate termites, or alternatively by laboratory synthesis of the desired compounds. Analogues of the natural materials, of the types described herein, could be obtained by chemical conversion from the natural materials, or alternatively by total synthesis in cases where such a route would be more efficaceous.

The synthesis of the basic tricyclic nucleus of the trinervitane diterpenes has been accomplished by means of Robinson annelation and McMurry coupling to yield oxygenated trinervitadiene products carrying olefinic functionality at 1(15),8(9)- or 1(15),8(19)-positions (Dauben et al., 1998). Furthermore, a trinervitatriene-2,3-diol carrying olefinic functionality at the 7(8),11(12),15(17)-positions has been synthesised by chemically simulating the proposed biogenetic route to such natural products (Hirukawa et al., 1994; Kato et al., 1998 and 2001). Extension or adaptation of these routes using chemical reactions well known and described in the art (cf., for example, Trost, 1989; ApSimson, 1973-1992), or the development of purpose designed synthetic routes again using chemical reactions well known and described in the art (cf., for example, Trost, 1989; ApSimson, 1973-1992), would provide convenient access to the natural trinervitanes and their analogues.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

The invention will hereinafter be further described by reference to the following non-limiting examples.

### EXAMPLE 1

### Methods and Materials

A sample comprising 11.59 g wet weight of Nasutitermes triodiae (Isoptera:Termitidae) (Froggatt) adults (mixed castes; mainly soldiers) was collected manually in the field and the sample was snap frozen in a dry-shipper containing liquid nitrogen. The specimen was stored at −80° C. before being freeze-dried to constant weight (1.39 g). The sample was ground to a powder and dispersed in 49 mL of 70% (v/v) methanol in water and shaken at room temperature overnight. The sample was filtered and centrifuged and the supernatant was recovered. The combined residues were then re-extracted with a further 20 mL of 70% methanol. The supernatants were combined to a total of 47 mL.

Antimicrobial activity was detected by saturating a ¼ inch diameter filter paper disk (Bacto) with the methanolic extract, evaporating the solvent in a cool air stream and placing the disk onto a bacteriological plate containing Bacillus subtilis which is known in the art to be a model organism for Gram positive bacteria (van Welt et al., 2001; Harwood et al., 1992; Ferreira et al., 2004). The Bacillus subtilis ATCC strain 6633 was used at 9.2 mL of a log phase culture with Abs₆₀ₙₘ=1 per 200 mL of Luria-Bertani medium containing 1.5% (w/v) agar. The plate was incubated at 28° C. for 24 hours and the diameter of the clearing zone was measured. Dilutions of the extract and fractions from HPLC chromatography were tested in the same way. In some cases, fractions from the column were tested by evaporating them to dryness, redissolving in methanol and simply spotting 10 μL of the methanolic sample directly onto the bacteriological plate and proceeding as described.

For the purification of the compound of fraction 23, (6) 6 mL of the methanolic extract was purified in 12×0.5 mL batches by semi-preparative reverse-phase HPLC over a YMC ODS-AQ capped C18 column (250 mm×10 mm) (Sapphire Biosystems) under the following conditions:

Loading conditions were:
0.5 mL of extract for each batch
Solvent A=99.95% water+0.05% (v/v) trifluoroacetic acid
Solvent B=100% acetonitrile

Elution conditions were:
0-2 minutes 100% A
2-22 minutes linear gradient 0-100% B
22-35 minutes 100% B
flow rate 4 mL/minute

Fractions were collected by time (1 minute per fraction). The absorbance of the effluent was monitored at 230 nm.

Corresponding fractions were pooled across all 12 batches and the eluate in each of the pooled fractions was evaporated to dryness under nitrogen. The residues were weighed and taken up again in small volumes of appropriate solvents—methanol for preparative or analytical HPLC and electrospray mass spectroscopy, deuterated chloroform for nuclear magnetic resonance (nmr) spectroscopy, etc.

For compounds in fractions other than fraction 23, a similar protocol was used but with the following additional steps. An extra 4 mL of extract was used and the eluates from all 20 batches were pooled and processed as described above. The material in fractions 24 and 26 was a mixture after this first preparative HPLC step, therefore the pooled active fractions were further purified using one of the two following isocratic chromatographic procedures.

In both isocratic purifications the same YMC ODS-AQ capped C18 column (250 mm×10 mm) (Sapphire Biosystems) was used as in the first step.

Isocratic Procedure 1

(Used to Purify Fraction 24, (7) in 4 Batches)

Loading conditions were:
for each batch, 0.5 mL of fraction 24 (7) in methanol

Elution conditions were:
Acetonitrile:tetrahydrofuran:water*=42:28:30 for 20 minutes
   Isocratic Procedure 2
   (Used to Purify Fraction 26, (8 and 9) in 2 Batches)

Loading conditions were:
for each batch 0.5 mL of fraction 26 (8 and 9) in methanol

Elution conditions were:
acetonitrile : water*=80:20 for 25 minutes
   (*water contained 0.05% trifluoroacetic acid (v/v))

The purified fractions were examined by analytical HPLC using similar gradient elution conditions to the preparative procedure, i.e. a water-acetonitrile gradient, followed by 100% acetonitrile. The only differences were that the analytical column was a YMC ODS-AQ capped C18 column (250 mm×3 mm), the flow rate was 0.55 mL/minute and 20 μL of sample was loaded onto the column for each run.

The purity and composition of the active pooled fractions were determined using a range of standard spectroscopic techniques including electrospray mass spectroscopy (ESMS), high resolution electron impact mass spectroscopy (HREIMS), electron impact mass spectroscopy (EIMS) and 300 and 500 MHz proton and carbon nuclear magnetic resonance in one and two dimensional modes.

Effects on mammalian cell growth were determined by exposing cultures of two mammalian neoplastic cell lines (SP2/0—Ag8, a non-secreting mouse myeloma cell line derived from Balb/C mice, and NCl-H460, a human-derived small cell lung carcinoma line) to fixed dilutions of the methanolic extract of N. triodiae or to fixed concentrations of fraction 23, (6) for 19 hours at 37° C. Cells were grown in wells of sterile 96-well tissue culture cluster plates by standard methods. Cell growth was estimated using the Cell Proliferation Reagent WST-1 (Roche Diagnostics) according to the manufacturer's instructions and proliferation data were compared with those from untreated control wells.

Minimum inhibitory concentrations with Bacillus subtilis ATCC strain 6633 were determined using the National Committee for Clinical Laboratory Standards broth microdilution test (NCCLS, 2000. NCCLS Document M7-A5—Methods for Dilution Antimicrobial Susceptibility Tests for Bacteria that Grow Aerobically, Approved Standard—Fifth Edition). The test was standardised using penicillin G and gentamicin with Staphyloccous aureus ATCC strains 29213 and 25923 and Enterococcus faecalis ATCC strain 29212. Results of standardisation were assessed according to the NCCLS standards (NCCLS, 2000. NCCLS Document M100-S10(M7)—Performance Standards for Antimicrobial Susceptibility Testing; Tenth Informational Supplement (Aerobic Dilution). NCCLS, Wayne, Pa.).

### RESULTS

The crude 70% methanol extract of N. triodiae displayed antimicrobial activity against B. subtilis (clear zone diameter 9 mm in the standard filter disk test) and moderate inhibitory activity against mammalian cells (37% of control at a concentration of approximately 30 μg/mL). There was no activity against a test strain (ACM 3221) of Escherichia coli (a Gram negative bacterium) in a similar test protocol.

After chromatographic fractionation of the methanolic activity, antimicrobial activity was detected in the fraction eluting between 22 and 23 minutes (fraction 23) and also the fractions eluting between 23 and 24 minutes (fraction 24) and between 25 and 26 minutes (fraction 26).

Fraction 23

A total of 9 mg of the pure compound was purified from 6 mL of crude extract, indicating a starting concentration of 1.5 mg/mL.

The molecular formula of the compound in fraction 23 (6) was determined as C₂₀H₃₂O₃ by ESMS which show sodiated ions at m/z 343 (MNa⁺) and m/z 663 (M₂Na⁺) and by HREIMS which showed M⁺-H₂O at m/z 302.2243 where m/z calculated for C₂₀H₃O₂ is 302.2246.

The ¹H NMR and ¹³C NMR chemical shift data for the compound (6) in fraction 23 are shown in Table 1. The compound in fraction 23 was determined to be 1(15),8(19)-trinervitadiene-3α,5α,18-triol (6). This compound has not been reported previously.

The minimum inhibitory concentration of compound (6) against B. subtilis was estimated as ≦50 μg/mL. Purified compound (6) had no detectable inhibitory effect on the proliferation of NCl-H460 cells at concentrations up to 100 μg/mL. Compound (6) had no detectable inhibitory effect on the proliferation of SP2/0 cells at concentrations up to 30 μg/mL. 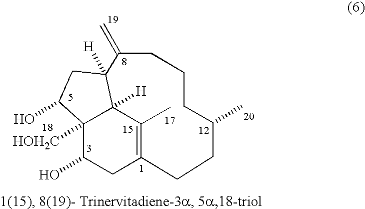
Fraction 24

The material in fraction 24, which also showed antimicrobial activity, was a mixture of at least two compounds after the first chromatographic step. It was therefore submitted to “Isocratic procedure 1” as described above and the single biologically active u.v.-absorbing peak which eluted between 14 and 17 minutes was collected.

A total of 4 mg of the pure biologically active compound present in fraction 24 was purified from 10 mL of starting material indicating an approximate starting concentration of 0.4 mg/mL in the crude extract.

The molecular formula of the compound in fraction 24 (7) was determined as C₂₀H₃₂O₂ by ESMS which show sodiated ions at m/z 327 (MNa⁺) and by HREIMS which showed M⁺ at m/z 304.2403 where m/z calculated for C₂₀H₃₂O₂ is 304.2402.

The ¹H NMR and ¹³C NMR chemical shift data for the compound (7) in fraction 24 are shown in Table 2. The compound in fraction 24 was determined to be 1(15),8(19)-trinervitadiene-3α,5α-diol (7). This compound has not been reported previously.

5 μg of compound (7) gave a clear zone of diameter 7 mm in the disc diffusion assay. The minimum inhibitory concentration of compound (7) against B. subtilis was estimated as ≦50 μg/mL. 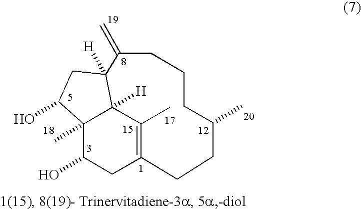
Fraction 26

The material in fraction 26 was a mixture of at least two biologically active compounds after the first chromatographic step. It was therefore submitted to “Isocratic procedure 2” as described above and two active fractions were collected. The first, which eluted from the column at approximately 12 minutes is designated Fraction 26A, the second, which eluted from the column at approximately 14 minutes is designated Fraction 26B.

Fraction 26A

A total of 0.5 mg of the pure biologically active compound present in fraction 26A was purified from 10 mL of starting material indicating an approximate starting concentration of 0.05 mg/mL in the crude extract.

The molecular formula of the compound in fraction 26A (8) was determined as C₂₂H₃₄O₄ by ESMS which showed ions at m/z 345 (MH⁺-H₂O) 363 (MH⁺), 385 (MNa⁺), 747 (M₂Na⁺) and by HREIMS which showed M⁺ at m/z 362.2460, where C₂₂H₃₄O₄ requires 362.2457; and M⁺-H₂O at m/z 344.2350 where C₂₂H₃₂O₃ requires 344.2351.

The ¹H NMR chemical shift data for the triol monoacetate (8) in fraction 26A are shown in Table 3. The identity of the triol monoacetate was further confirmed by partially acetylating the triol (6) and confirming the presence in the acetylation mixture of a major component with identical retention time and ¹H NMR spectrum to the natural triol monoacetate. The protocol used was as follows:

The triol (6) (1 mg) and acetic anhydride (10 μL) in dry pyridine (100 μL) were kept for 3 hours at room temperature. The reaction was diluted with water, extracted with dichloromethane, and the extract subjected to preparative HPLC on the standard column using gradient elution in acetonitrile/water from 50:50 to 100:0. The fraction with retention time 18 min when analysed under normal gradient elution conditions contained a major component with identical retention time to the natural triol monoacetate (8). The ¹H NMR spectrum of this major component (Table 3) also matched that of the natural triol monoacetate (8).

The compound in fraction 26A was therefore determined to be 1(15),8(19)-trinervitadiene-3α,5α,18-triol 5-acetate (8). This compound has not been reported previously.

An unknown concentration and mass of the compound (8) gave a clear zone of diameter 15 mm in the disc diffusion assay. The minimum inhibitory concentration of compound (8) in the standard broth microdilution assay against B. subtilis was estimated as >50 μg/mL. 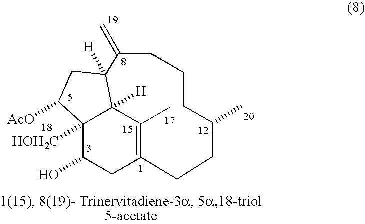
Fraction 26B

A total of 1.5 mg of the pure biologically active compound present in fraction 26B was purified from 10 mL of starting material indicating an approximate starting concentration of 0.15 mg/mL in the crude extract.

The molecular formula of the compound in fraction 26B (9) was determined as C₂₀H₃₂O₂ by ESMS which showed sodiated ions at m/z 327 (MNa⁺), 631 (M₂Na⁺) and by HREIMS which showed M⁺ at m/z 304.2404 where C₂₀H₃₂O₂ requires 304.2402.

The ¹H NMR chemical shift data for the trinervitadiene diol (9) in fraction 26B are shown in Table 4. The compound in fraction 26B was therefore determined to be 1(15),8(9)-trinervitadiene-2β,3α-diol (9) by comparison of the measured chemical shifts with previously published ¹H NMR data (Goh, Chuah et al., 1984; Braekman, Daloze et al., 1983; Prestwich & Collins, 1981; Prestwich et al., 1976b) for this compound (Table 4).

An unknown concentration and mass of the compound (9) gave a clear zone of diameter 15 mm in the disc diffusion assay. The minimum inhibitory concentration of compound (9) in the standard broth microdilution assay against B. subtilis was estimated as ≦25 μg/mL. Although the structure of this compound has been published previously, it has not previously (e.g. Goh, Chuah et al., 1984; Braekman, Daloze et al., 1983; Prestwich & Collins, 1981; Prestwich et al., 1976b) been reported that it has potent antimicrobial activity. 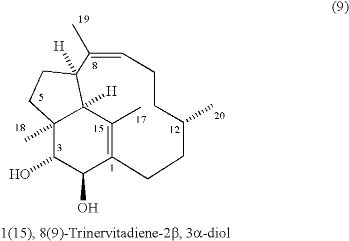
1(15),8(19)-Trinervitadiene-3α,5α,18-triol 3,5,18-Triactate (10)

1(15),8(19)-Trinervitadiene-3α,5α,18-triol 3,5,18-triacetate (10) was synthesised by acetylation of the triol (6). The triol (6) (1 mg) and acetic anhydride (30 μL) in dry pyridine (100 μL) were kept for 3 days at room temperature. The reaction was diluted with water, extracted with dichloromethane, and the extract subjected to preparative HPLC under the standard conditions. The major fraction was collected and filtered through a short silica column in dichloromethane to afford the triacetate (10) (1 mg), which eluted as a single major peak at 28 minutes when chromatographed under the standard analytical HPLC conditions described in the above Materials and Methods section.

The molecular formula of the reaction product was confirmed as C₂₆H₃₈O₆ by ESMS which showed sodiated ions at m/z 469 (MNa⁺) and by HREIMS which showed (M⁺-AcOH) at m/z 386.2449 where C₂₄H₃₄O₄ requires 386.2457.

The ¹H NMR chemical shift data for the trinervitadiene triol triacetate (10) reaction product are shown in Table 5. The acetylation product was therefore determined to be 1(15),8(19)-trinervitadiene-3α,5α,18-triol 3,5,18-triacetate (10). This compound has not been reported previously.

5 μg of compound (10) gave a clear zone of diameter 11 mm in the disc diffusion assay. The minimum inhibitory concentration of compound (10) against B. subtilis was estimated as >50 μg/mL.

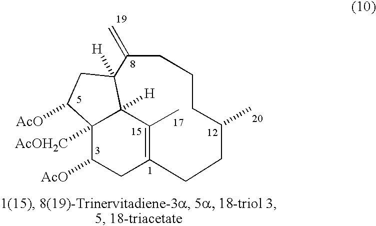

### EXAMPLE 2

In studies on the antimicrobial activity of other Australian termites of the genus Nasutitermes (Family: Termitidae; Sub-Family: Nasutitermitinae) it was noted that extracts of three other species, Nasutitermes exitiosus (Hill) and two unidentified species of the same genus, exhibited antimicrobial activity which eluted at the same retention time as 1(15),8(9)-Trinervitadiene-2β,3α-diol (9) and shared identical ¹H NMR chemical shift data (Table 4) with the diol (9) purified from fraction 26B.

It was also noted that extracts made exclusively from workers of the species N. exitiosus exhibited no antimicrobial activity and did not exhibit any of the characteristic u.v. absorbing peaks attributed to trinervitadiene derivatives, which elute in the 22-28 minute region of the standard gradient HPLC chromatogram. On the other hand, an extract of soldiers from the same nest as the aforementioned workers exhibited antimicrobial activity.

When the extract of soldier termites was subjected to the standard gradient HPLC chromatography, the biologically active u.v.-absorbing peaks characteristic of trinervitadiene derivatives were observed. This indicates that separation of soldier termites from workers prior to their extraction may improve the efficiency both of detecting and purifying biologically active trinervitadiene derivatives.

Discussion:

A range of compounds with the trinervitadiene carbon skeleton have previously been reported from termites (for example: Prestwich, Tanis et al. 1976a,b; Vrkoc, Budesinsky et al. 1978a,b; Dupont, Braekman et al. 1981; Prestwich, Spanton et al, 1981; Baker & Walmsley, 1982; Braekman, Daloze et al. 1986). Trinervitadiene derivatives have been found in extracts of a number of species of termites belonging to nine genera of termites within the subfamily Nasutitermitinae of the family Termitidae. However, this is the first time that the isolation of compounds (6, 7, and 8) have been reported or that the triacetate(10) of compound (6) has been prepared. The novelty of these compounds is underlined by the fact that no trinervitadiene derivatives have previously been reported with hydroxylation or other substitutions at positions 5 and 18 on the carbon skeleton (e.g. compound (6)). Furthermore, no data have been reported previously regarding antimicrobial activity of any trinervitadiene or derivative thereof including the compounds whose isolation or preparation is described herein (6, 7, 8, 9 and 10).

It has been determined that compound (6) has a minimum inhibitory concentration in the range≦50 parts per million and that it is at least twice as toxic to microbial cells as it is to human cells and potentially significantly more selective than this. Compound (9) has a minimum inhibitory concentration in the range of ≦25 parts per million and has significant inhibitory activity as low as 12 parts per million. Compounds (7), (8) and (10) have reduced but detectable antimicrobial activity. For example, acetylation of the hydroxyl groups seems to reduce but not abolish activity, whilst it is also clear that the number and arrangement of groups on the trinervitadiene carbon skeleton modulates the level of antimicrobial activity.

Compounds (6, 7, 8, 9 and 10) and a range of other trinervitadiene derivatives where the number, position and nature of groups is varied (such as the specified derivatives of compounds 11-16) can therefore be expected to have utility as antibiotics or for some of the other purposes mentioned above. Alternatively, they may be useful lead compounds for the development of derivatives with enhanced antibiotic activities.

It is interesting that prolonged investigations of the role of soldier defensive secretions has led to the classification of the trinervitadiene derivatives as “defensive compounds” (i.e. the assumption has been that their function is solely in defence of the termite colony against attack by invertebrate or vertebrate predators). However, the present discovery that these compounds have antimicrobial activity raises the possibility that they also function naturally to suppress microbial parasites within the termite colony.

TABLE 1
1(15),8(19)-Trinervitadiene-3α,5α,18-triol (6)
¹H and ¹³C NMR data (CDCl₃):

| Position | δ C^{a} | δ H^{b,c} |
|---|---|---|
| 1 | 127.9 | — |
| 2 | 36.6 | 2.40(m), 2.18(m) |
| 3 | 70.1 | 4.16(bs) |
| 4 | 53.3 | — |
| 5 | 74.8 | 4.68(d, 3.5) |
| 6 | 36.3 | 2.18(m), 1.84(m) |
| 7 | 49.2 | 3.47(m) |
| 8 | 149.7 | — |
| 9 | 27.2 | 1.92(m)1.83(m), |
| 10 | 23.8 | 1.59(m), 1.59(m) |
| 11 | 31.9 | 1.21(m), 0.91(m) |
| 12 | 27.3 | 1.31(m) |
| 13 | 32.0 | 1.43(m), 1.43(m) |
| 14 | 27.8 | 2.40(m), 1.68(m) |
| 15 | 126.4 | — |
| 16 | 51.9 | 2.55(d, 11.5) |
| 17 | 21.0 | 1.68(s) |
| 18 | 64.2 | 3.95(d, 11.0), 3.80(bs) |
| 19 | 113.3 | 4.99(s), 4.86(s) |
| 20 | 21.8 | 0.88(d, 7.0) |

^{a}Chemical shifts, (δ) with CDCl₃(76.9) as reference for solutions in CDCl₃ at 75.43MHz.
^{b}Chemical shifts (δ) with CHCl₃(7.26) as reference for solutions in CDCl₃ at 500Mhz.
^{c}Where individual proton multiplets are unresolved chemical shifts are approximate.

TABLE 2
1(15),8(19)-Trinervitadiene-3α,5α-diol (7)
¹H and ¹³C NMR data (CDCl₃):

| Position | δ C | δ H |
|---|---|---|
| 1 | 127.7 | — |
| 2 | 36.8 | 2.29(dd, 6, 16.5) |
| | | 1.95(m) |
| 3 | 68.7 | 3.97(dd, 11.0, 6.5) |
| 4 | 49.9 | — |
| 5 | 76.9 | 4.27(d, 4) |
| 6 | 36.6 | 2.16(m), 1.75(m) |
| 7 | 49.0 | 3.46(m) |
| 8 | 150.2 | — |
| 9 | 27.0 | 1.98(m), 1.80(m) |
| 10 | 23.9 | 1.58(m), 1.58(m) |
| 11 | 32.0 | 1.20(m), 0.93(m) |
| 12 | 27.2 | 1.36(m) |
| 13 | 32.1 | 1.36(m), 1.44(m) |
| 14 | 27.9 | 2.41(m), 1.70(m) |
| 15 | 126.7 | — |
| 16 | 55.6 | 2.70(d, 10.5) |
| 17 | 21.2 | 1.71(s) |
| 18 | 12.4 | 0.98(s) |
| 19 | 112.9 | 4.98(s), 4.85(s) |
| 20 | 21.8 | 0.89(d, 6.5) |

TABLE 3
1(15),8(19)-Trinervitadiene-3α,5α,18-triol 5-acetate (8)
¹H NMR data (CDCl₃):

| | Natural acetate | Acetylation product |
|---|---|---|
| Position | δ H | δ H |
| H-3 | 4.14(m) | 4.14(m) |
| H-5 | 5.72(d, 4.0) | 5.72(d, 4.5) |
| H-7 | 3.40(m) | 3.40(m) |
| H-17 | 1.67(s) | 1.67(s) |
| H-18 | 3.85(d, 13.0) | 3.85(d, 13.0) |
| | 3.40(d, 13.0) | 3.40(m) |
| H-19 | 5.02(d, 2.0) | 5.02(d, 2.0) |
| | 4.90(d, 2.0) | 4.90(d, 2.0) |
| H-20 | 0.8(d, 6.0) | 0.89 |
| OCOCH₃ | 2.18(s) | 2.17(s) |

TABLE 4
1(15),8(9)-Trinervitadiene-2β,3α-diol (9)
¹H NMR data (CDCl₃):

| | | Goh, | | | | |
|---|---|---|---|---|---|---|
| | | Chuah | Braekman, | Prestwich | | |
| | This | et al. | Daloze et al. | & Collins | Prestwich | |
| | patent | (1984) | (1983) | (1981) | et al. | |
| Position | δH | (CDCl₃) | (CDCl₃) | (CDCl₃) | (1976b) | Group |
| H-2 | 4.03(d, 8) | 4.0(d) | 4.05(d, 10) | 4.05(br, d, 8) | | CHOH |
| H-3 | 3.71(d, 9) | 3.76(d) | 3.70(d, 10) | 3.70(d, 8.5) | | CHOH |
| H-9 | 5.29(dd 11.0, | | 5.30(dd, 10, 5) | 5.30(br, m) | 5.28(ddq, 12, 6, | CH═ |
| | 6.0) | | | | 1.8) | |
| H-17 | 1.69(br) | 1.67(d) | 1.68(bs) | 1.69(d, 0.6) | | CH₃ |
| H-18 | 0.97(s) | 0.97(s) | 0.95(s) | 0.99(s) | | CH₃ |
| H-19 | 1.56(d, 2.0) | 1.49(s) | 1.58(d) | 1.57(d, 1.2) | 1.59(d, 1.8) | CH₃ |
| H-20 | 0.85(d, 7.0) | 0.90(d, 6.6) | 0.85(d, 6) | 0.86(d, | | CH₃ |
| | | | | 6.1) | | |

TABLE 5
1(15),8(19)-Trinervitadiene-3α,5α,18-triol 3,5,18-triacetate (10)
¹H NMR data (CDCl₃):

| Position | δ H |
|---|---|
| 3 | 5.36(dd, 11.0, 6.5) |
| 5 | 5.22(d, 4.0) |
| 7 | 3.43(m) |
| 16 | 2.90(d, 12.0)) |
| 17 | 1.71(s) |
| 18 | 4.54(d, 11.5) |
| | 4.04(d, 11.5) |
| 19 | 5.02(s), 4.92(s) |
| 20 | 0.89(d, 6.5) |
| OCOCH₃ | 2.09(s) |
| | 2.06(s) |
| | 1.98(s) |

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

ApSimon, J. (Editor) The Total Synthesis of Natural Products, Vols. 1-9, Wiley and Sons: New York, 1973-1992.
Baker, R., S. Walmsley (1982) “Soldier defense secretions of the South American termites Cortaritermes silvestri, Nasutitermes sp N.D. and Nasutitermes kemneri.” Tetrahedron 38(13): 1899-1910.
Braekman, J. C, D. Daloze, et al. (1983). “Chemical composition of the frontal gland secretion from soldiers of Nasutitermes lujae (Termitidae, Nasutitermitinae).” Tetrahedron 39(24): 4237-4241.
Braekman, J. C., D. Daloze, et al. (1986). “Two new C-20 substituted trinervitane diterpenes from a Neo-Guinean Nasutitermes species” Bulletin of the Chemical Society of Belgium 95(9-10): 915-919.
Dauben, W. G., Lorenz, K. L., Dean, D. W., Shapiro, G., Farkas, I. (1998) Tet. Letters 39, 7079-7082.
DeCoursey, J. D., A. P. Webster, et al. (1953). “An antibacterial agent from Tribolium castaneum (Herbst).” Annals of the Entomological Society of America 46: 386-392.
Dupont, A., J. C. Braekman, et al. (1981). “Chemical composition of the frontal gland secretions from Neo-Guinean nasute termite soldiers.” Bulletin of the Chemical Society of Belgium 90(5): 485-499.
Ferreira, I. C. F. R., Calhelha, R. C., Estevinho, L. M. and Queiroz, M-J. R. P. (2004) “Screening of antimicrobial acticity of diarylamines in the 2,35-tri-methylbenzo[b]thiophene series: a structure-activity evaluation study.” Bioorganic & Medicinal Chemistry Letters 14: 5831-5833.
Goh, S. H., C. H. Chuah, et al. (1984). “Extreme intraspecific chemical variability in soldier defense secretions of allopatric and sympatric colonies of Longipeditermes longipes.” J. Chem. Ecol. 10(6): 929-944.
Harwood, C. R. (1992) “Bacillus subtilis and its relatives: molecular biological and industrial workhorses.” TIBTECH 10: 247-256.
Hirukawa, T.; Suzuki, T.; Tanaka, M.; Kato, T. (1994) J. Chem. Soc., Chem. Commun, 311-312.
Kato, T., Hirukawa, T., Suzuki, T., Tanaka, M., Hoshikawa, M., Yagi, M., Tanaka, M., Takagi, S. S., Saito, N. (2001) Helv. Chim. Acta 84, 47-68.
Kato, T., Tanaka, M., Hoshikawa, M., Yagi, M. (1998) Tet. Letters 39, 7553-7556.
Leem, J. Y., I. J. Jeong, et al, (1999). “Isolation of p-hydroxycinnamaldehyde as an antibacterial substance from the saw fly Acantholyda parki S.” FEBS Letters 442: 53-56.
Prestwich, G. D., M. S. Collins (1981). “Chemotaxonomy of Subulitermes and Nasutitermes termite soldier defense secretions. Evidence against the hypothesis of diphyletic evolution of the Nasutitermitinae.” Biochem. Syst. Ecol. 9(1): 83-88.
Prestwich, G. D., S. G. Spanton, et al. (1981). “New tricyclic diterpene propionate esters from a termite soldier defense secretion.” Tetrahedron Letters 22(17): 1563-1566.
Prestwich, G. D., P. Tanis, et al. (1976a). “Nasute termite soldier frontal gland secretions. 1. Structure of trinervi-2β,3α,9α-triol 9-O-Acetate, a novel diterpene from Trinervitermes soldiers.” Journal of the American Chemical Society 98(19): 6061-6062.
Prestwich, G. D., S. P. Tanis, et al. (1976b). “Nasute termite soldier frontal gland secretions. 2. Structures of trinervitene congeners from Trinervitermes soldiers.” Journal of the American Chemical Society 98(19): 6062-6064.
Trost, B. M. (Editor-in-Chief) Comprehensive Organic Synthesis. Selectivity, Strategy and Efficiency in Modern Organic Chemistry, Vols 1-9; Pergamon Press: Oxford, 1991.
van Welt, K. H. M., Swaving, J., Freudl, R. and Driessen, A. J. M. (2001) “Translocation of proteins across the cell envelope of Gram-positive bacteria.” FEMS Microbiology Reviews 25, 437-454.
Vrkoc, J., M. Budesinsky, et al. (1978a). “Structure of 2α,3α-dihydroxy- and 2α,3β-dihydroxy-1(15),8(19)-trinervitadienes from Nasutitermes costalis (Holmgren).” Collection of Czechoslovak Chemical Communications 43: 2478-2485.
Vrkoc, J., M. Budesinsky, et al. (1978b). “Structure of trinervitene diterpenoids from Nasutitermes rippertii (Rambur).” Collection of Czechoslovak Chemical Communications 43: 1125-1132.

1-98. (canceled)

99. A pharmaceutical and/or veterinary formulation, said formulation comprising a compound according to the formula: 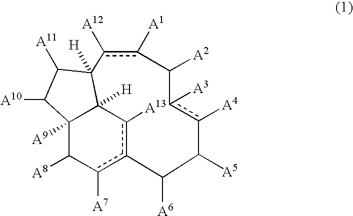
wherein; [symbol]
denotes a single or double bond or an epoxidised bond, and
substituents A¹ to A¹³ are selected, independently from the group consisting of H, OH, O, SH, NH₂, lower alkyl, lower alkene, lower alkoxy, lower carboxy, lower aldehyde groups, lower ketone groups, lower ester groups, lower acyloxy, lower alkoxyalkyl, lower alcohol groups or lower alkylamino,
or pharmaceutical/veterinary-acceptable salt thereof,
in admixture with a suitable pharmaceutically/veterinary-acceptable excipient.

100. The formulation of claim 99, wherein A¹, A², A³, A⁵, A⁶, A⁷, A⁸, A¹⁰ and A¹¹ are selected, independently, from the group consisting of H, OH, O, SH, NH₂ and OR, where R in the group OR is a lower alkyl or lower acyl.

101. The formulation of claim 99, wherein A⁴, A⁹ and A¹³ are selected, independently, from the group consisting of lower alkyl, lower carboxy, lower aldehyde, lower ketone, lower ester, lower acyloxy or lower alcohol.

102. The formulation of claim 101, wherein A⁴ and A¹³ are selected, independently, from the group consisting of methyl, methanoate and methanol, A⁹ is selected from methanol and CH₂OR groups, where R in the group OR is a lower alkyl or lower acyl.

103. The formulation of claim 99, wherein A¹² is lower alkyl or lower alkene.

104. The formulation of claim 99, wherein at least two of said A¹ to A¹³ are a OH or OR group, and R in the group OR is a lower alkyl.

105. The formulation of claim 99, wherein the compound is of the formula: 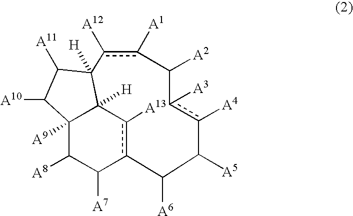
wherein; [symbol]
denotes a single or double bond or an epoxidised bond, and
substituents A¹ to A¹³ are selected, independently, from the group consisting of H, OH, O, lower alkyl, lower alkene, lower alkoxy, lower carboxy, lower aldehyde, lower ketone, lower ester, lower acyloxy and lower alcohol; or pharmaceutically/veterinary acceptable salts thereof.

106. The formulation of claim 105, wherein at least two of said A¹ to A¹³ are a OH or OR group, and R in the group OR is a lower alkyl.

107. The formulation of claim 99, wherein the compound is of the formula: 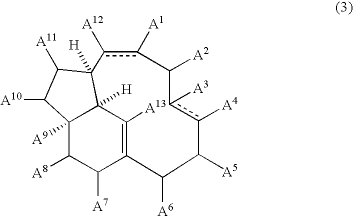
wherein; [symbol]
denotes a single or double bond or an epoxidised bond, and
substituents A¹ to A¹³ are selected, independently, from the group consisting of H, OH, O, methyl, ethyl, propyl, butyl, methene, ethene, propene, methanal, ethanal, propanal, butanal, methanoate, ethanoate, propanoate, butanoate, acetoxy, propionyloxy, butyryloxy, methanol, ethanol, propanol and butanol; or pharmaceutically/veterinary acceptable salts thereof.

108. The formulation of claim 107, wherein at least two of said A¹ to A¹³ are a OH or OR group, where R in the group OR is a lower alkyl.

109. The formulation of claim 99, wherein the compound is of the formula: 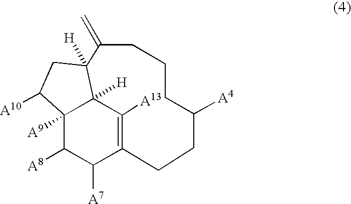
wherein;
substituents A⁴, A⁷, A⁸, A⁹, A¹⁰ and A¹³ are as defined in claim 99;
or pharmaceutically/veterinary acceptable salts thereof.

110. The formulation of claim 99, wherein the compound is of the formula: 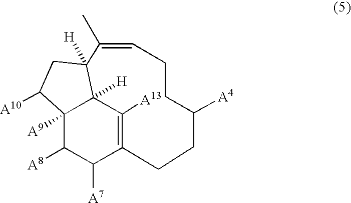
wherein;
substituents A⁴, A⁷, A⁸, A⁹, A¹⁰ and A¹³ are as defined in claim 99;
or pharmaceutically/veterinary acceptable salts thereof.

111. The formulation of claim 110, wherein A⁴, A⁷, A⁸ and A¹⁰ are selected, independently, from the group consisting of H, OH, O, SH, NH₂ and OR, where R in the group OR is a lower alkyl or lower acyl.

112. The formulation of claim 110, wherein A⁹ and A¹³ are selected, independently, from the group consisting of lower alkyl, lower alkoxy, lower carboxy, lower aldehyde, lower ketone, lower ester, lower acyloxy and lower alcohol.

113. The formulation of claim 112, wherein A⁹ and A¹³ are selected, independently, from methanol or CH₂OR where R in the group OR is a lower alkyl or lower acyl.

114. The formulation of claim 110, wherein at least two of said A⁴, A⁷, A⁸, A⁹, A¹⁰ and A¹³ are a OH or OR group where R in the group OR is a lower alkyl.

115. The formulation of claim 99, wherein the compound is selected from the group consisting of;
1(15),8(19)-Trinervitadiene-3α,5α,18-triol,
1(15),8(19)-Trinervitadiene-3α,5α-diol,
1(15),8(19)-Trinervitadiene-3α,5α,18-triol 5-acetate,
1(15),8(9)-Trinervitadiene-2β,3α-diol, or
1(15),8(19)-Trinervitadiene-3α,5α,18-triol 3,5,18-triacetate.

116. A method for treating a Gram positive bacterial infection in a subject, said method comprising administering to said subject an effective amount of a compound according to the formula: 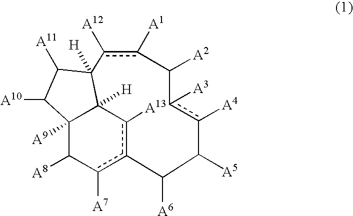
wherein; [symbol]
denotes a single or double bond or an epoxidised bond, and
substituents A¹ to A¹³ are selected, independently from the group consisting of H, OH, O, SH, NH₂, lower alkyl, lower alkene, lower alkoxy, lower carboxy, lower aldehyde groups, lower ketone groups, lower ester groups, lower acyloxy, lower alkoxyalkyl, lower alcohol groups or lower alkylamino, or pharmaceutically/veterinary acceptable salts thereof.

117. A method for disinfecting a surface, said method comprising applying to said surface an amount of a compound according to the formula: 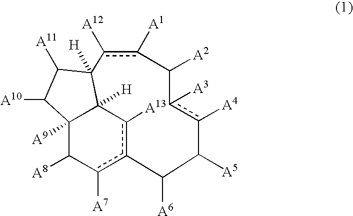
wherein; [symbol]
denotes a single or double bond or an epoxidised bond, and
substituents A¹ to A¹³ are selected, independently from the group consisting of H, OH, O, SH, NH₂, lower alkyl, lower alkene, lower alkoxy, lower carboxy, lower aldehyde groups, lower ketone groups, lower ester groups, lower acyloxy, lower alkoxyalkyl, lower alcohol groups or lower alkylamino, or pharmaceutically/veterinary acceptable salts thereof.
